(19) United States
(12) Patent Application Publication
(10) Pub. No.: US 2026/0294677 A1
(43) Pub. Date: Oct. 1, 2026

(54) COOLING APPARATUSES AND RELATED METHODS

(71) Applicant: THERMAL OVERWATCH LLC, Chapel Hill, NC (US)

(72) Inventors: Keller Balsam, Durham, NC (US); Joshua Heebner, Chapel Hill, NC (US)

(21) Appl. No.: 19/629,775

(22) Filed: Mar. 26, 2026

Related U.S. Application Data
(60) Provisional application No. 63/778,051, filed on Mar. 26, 2025.
(60) Provisional application No. 63/805,109, filed on May 13, 2025.

(51) Int. Cl. A61F 7/02

(52) U.S. Cl. CPC A61F 7/0241

(57) ABSTRACT

A cooling apparatus includes a housing, a rack supported within the housing having a receptacle, and a handle removably coupled to the receptacle. The handle includes a clamping mechanism for selectively clamping a fabric.

### CROSS-REFERENCE TO RELATED APPLICATIONS

This application claims priority to U.S. Provisional Patent Application No. 63/805,109 filed on May 13, 2025, and U.S. Provisional Patent Application No. 63/778,051 filed on Mar. 26, 2025, the entire contents of which are incorporated herein by reference.

### FIELD

The present disclosure relates to a cooling apparatus, and more particularly to a cooling apparatus for retaining cooling sheets and related methods.

### BACKGROUND

Heat stroke, or other heat related maladies, can be a medical emergency requiring rapid cooling. Conventional cooling methods can include wrapping a patient in bed linens soaked in ice water in order to reduce the patient's core temperature. Current methods of storing and retrieving these bed linens often involves reaching into a cooler to pull out the submerged soaked bed linens. However, users often find the bed linens tangled within the cooler, which can make it hard for the user, e.g., to identify corners of the bed linen for effective placement on the patient. Such disorganization can impact cooling performance of the bed linen on the patient, as well as slow down response time of treating the patient in emergency situations.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Further, the claimed subject matter is not limited to implementations that solve any or all disadvantages noted in any part of this disclosure.

Therefore, there exists a need for a cooling apparatus for emergency heat casualties, or other heat related maladies, that can provide rapid, easily accessible cooling methods in military, athletic, emergency, and/or field applications

The present subject matter relates to cooling apparatuses and related methods. More specifically, the present disclosure relates to support systems for cooling apparatuses used for treating emergency heat casualties, or other heat related maladies. Methods related to the manufacture and use of such cooling apparatuses as disclosed herein are also provided.

The present disclosure provides, in one aspect, a cooling apparatus that includes a housing, a rack supported within the housing that has a receptacle, and a handle removably coupled to the receptacle.

Thus, it is an object of the presently disclosed subject matter to provide cooling apparatuses as well as methods related thereto. While one or more objects of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 is a perspective view of a cooling apparatus according to an embodiment of the present disclosure.

FIG. 2 is a side perspective view of the cooling apparatus of FIG. 1.

FIG. 3 is a perspective view of embodiments of different racks of the cooling apparatus of FIG. 1 according to the present disclosure.

FIG. 4 is a perspective view of an embodiment of a handle of the cooling apparatus of FIG. 1 according to the present disclosure.

FIG. 5 is a side view of the handle of FIG. 4.

FIG. 6 is a perspective view of another embodiment of a cooling apparatus.

FIG. 7 is a perspective view of a conventional cooler containing cooling sheets used in a method of cooling an overheated person.

FIG. 8 is a perspective view of a method of cooling.

FIG. 9 is a perspective view of a patient wrapped in one or more cooling sheets using the method of cooling of FIG. 8.

FIG. 10 is a perspective view of another embodiment of a cooling apparatus.

FIG. 11 is an enlarged, partial perspective view of the cooling apparatus of FIG. 10.

FIG. 12 is a perspective view of an embodiment of a handle of the cooling apparatus of FIG. 10 according to the present disclosure.

FIG. 13 is a top perspective view of the cooling apparatus of FIG. 10 with one or more cooling sheets therein.

FIG. 14 is a side perspective view of the cooling apparatus of FIG. 10 according to another embodiment of the present disclosure.

FIG. 15 is a top view of the cooling apparatus of FIG. 14 showing an embodiment of a cover of the cooling apparatus according to the present disclosure.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

Other documentation and related information are provided herewith to further illustrate these and other aspects of the subject matter disclosed herein and to further provide disclosure that is enabling to one of ordinary skill in the art.

### DETAILED DESCRIPTION

Reference now will be made to the embodiments of the present subject matter, one or more examples of which are set forth below. Each example is provided by way of an explanation of the present subject matter, not as a limitation. In fact, it will be apparent to those skilled in the art that various modifications and variations can be made in the present subject matter without departing from the scope or spirit of the present subject matter. For instance, features illustrated or described as one embodiment can be used on another embodiment to yield still a further embodiment. It is to be understood by one of ordinary skill in the art that the present discussion is a description of exemplary embodiments only, and is not intended as limiting the broader aspects of the present subject matter, which broader aspects are embodied in exemplary constructions.

As used herein, the term a “plurality” means two or more.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “or” can be conjunctive or disjunctive.

As used herein, the term “substantially” means to a great or significant extent, but not necessarily completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

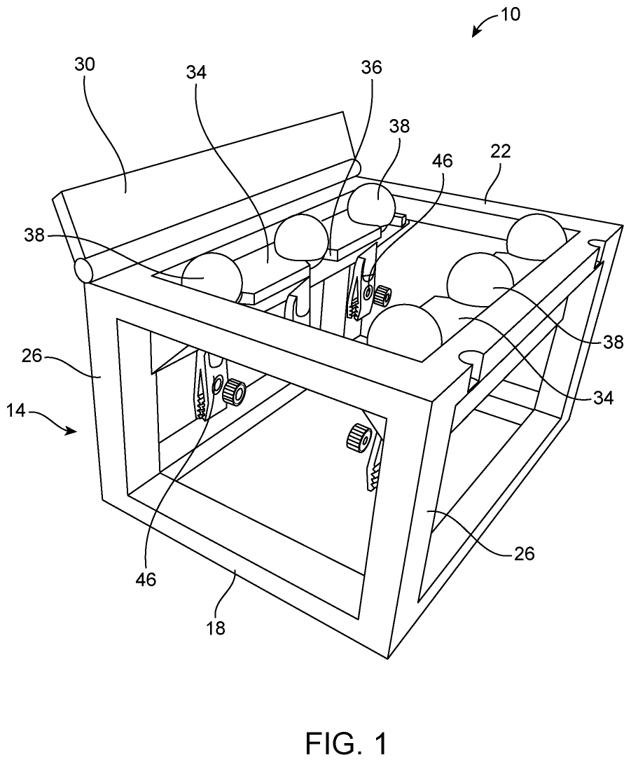

FIG. 1 illustrates a cooling apparatus. In the illustrated embodiment, the cooling apparatus is an insulative container 10, such as a cooler. As shown, portions of the side walls are removed to provide better viewing of the interior of the insulative container 10. In other embodiments, the cooling apparatus may be another type of insulative container. In other embodiments, the cooling apparatus may be part of a cooling system.

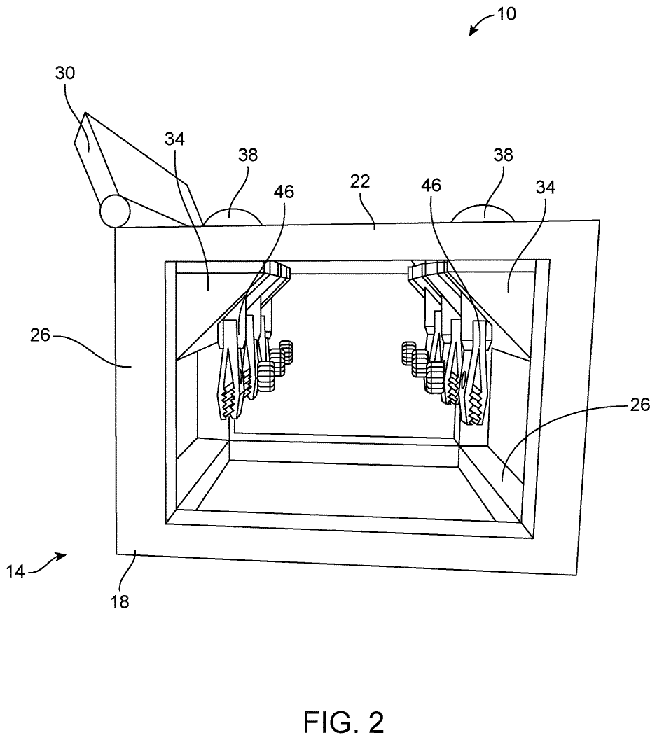

With reference to FIGS. 1-3, the illustrated container 10 includes a main body or housing 14 having a base 18 for supporting the container 10, an upper side 22 opposite the base 18, and sidewalls 26. The housing 14 defines an internal space therein for maintaining a constant temperature. To maintain the constant temperature, the illustrated embodiment includes a cover 30 that can be pivotably coupled to the upper side 22 of the housing 14. In other embodiments, the cover 30 can be coupled to the upper side 22 of the housing 14 in other manners. In some embodiments, the cover 30 can be removable from and securable to the upper side 22 of the housing 14.

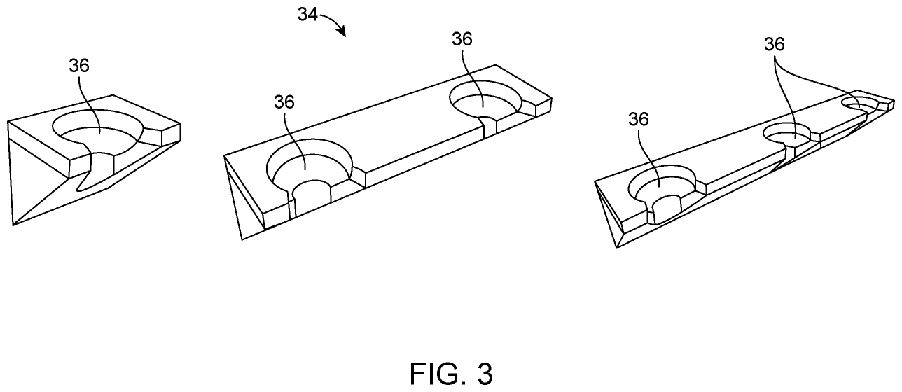

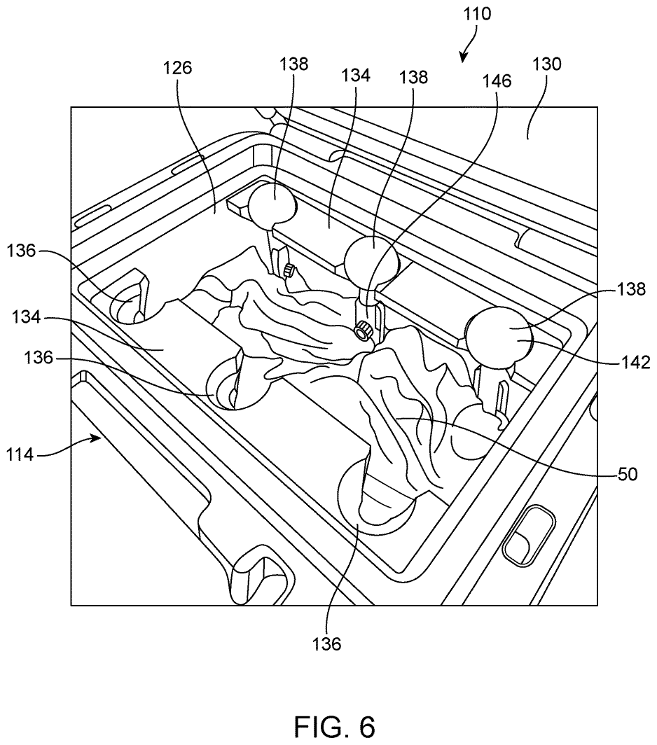

The housing 14 can be configured to support one or more racks 34. The illustrated embodiment includes two racks 34; however, other embodiments may include greater or fewer numbers of racks 34. In the illustrated embodiment, the racks 34 are integrated with respective sidewalls 26 of the housing 14. In other embodiments, the racks 34 are not integrated with the housing 14. Each of the illustrated racks 34 includes one or more receptacles 36 (FIG. 3) that are each configured to receive a handle 38 to support or mount the handle 38 on the rack 34. The receptacles 36 are shaped to retain the respective handles 38 in an upright position. In some embodiments as shown, the receptacles 36 can retain the respective handles 38 in an upright position substantially perpendicular to the base 18 of the housing 14. This orientation ensures the handles 38 remain in a consistent position during transport and use of the apparatus to prevent any sheets 50 (FIG. 6) from getting tangled, as explained in more detail below. In some embodiments, the handles 38 can be retained in respective receptacles 36 via a snap-fit connection. In some embodiments, the handles 38 can be retained in respective receptacles 36 via a fastener, such as a mechanical fastener or an adhesive substrate. In other embodiments, the cover 30 of the container 10 can include cut-outs and/or protrusions shaped to retain the handles 38 within the receptacles 36 during transport of the apparatus.

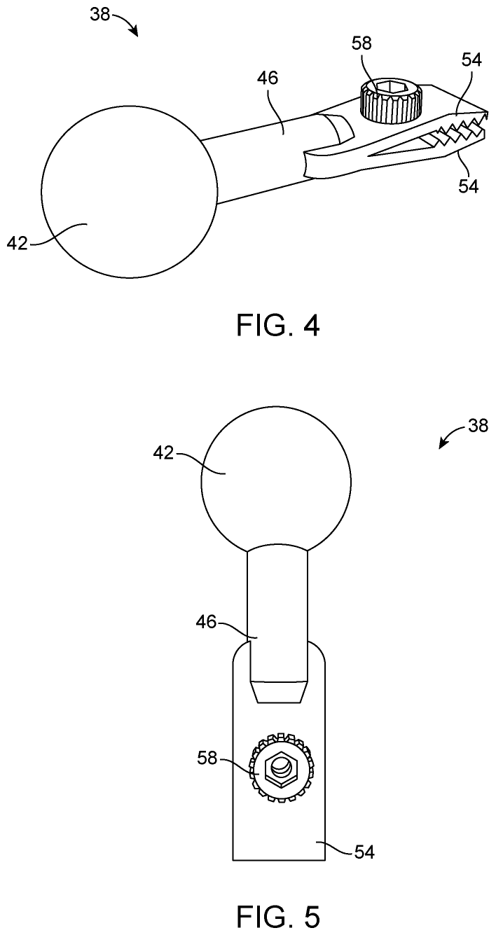

With reference to FIGS. 4-5, each of the handles 38 includes a gripping part 42 configured to be graspable by a user, and a clamping mechanism, such as a clamp 46, for selectively clamping a fabric, for example, a sheet 50 (e.g., a fabric bed sheet; FIG. 6). In the illustrated embodiment, the gripping part 42 is substantially spherical in shape. In other embodiments, the gripping part 42 may be other shapes. Additionally, the illustrated clamping mechanism 46 is integrated with the gripping part 42. In other embodiments, the clamping mechanism 46 can be securable to but not integrated with the gripping part 42. In yet other embodiments, the sheet 50 is directly attached to the rack 34 via an integrated clamping mechanism, such as a clip or other fastening means.

The illustrated clamping mechanism 46 for each of the handles 38 includes a plurality of jaws 54 and a fastener 58. The fastener 58 is configured to selectively loosen and tighten the jaws 54. For example, in the illustrated embodiment, the fastener 58 is a threaded fastener configured to threadably engage the jaws 54 to selectively open and close the jaws 54. In other embodiments, the fastener 58 can loosen or tighten the jaws 54 in other manners. In yet other embodiments, the clamping mechanism 46 is a clip, such as a tarp clip or a cloth clip.

Returning to FIGS. 1-3, during use, the container 10 is configured to hold a volume of fluid (e.g., water) within the housing 14 to saturate the sheets 50. In the illustrated embodiment, the sheets 50 are fabric bed sheets. In other embodiments, the sheets 50 are comprised of cloth created with other natural or synthetic fibers that are absorbent. The fluid can be configured to maintain the sheets 50 at a constant, cool temperature within the container 10. The fluid, for example, can be iced water, i.e., water with ice initially included within the water. To keep the sheets 50 saturated within the volume of the fluid, the internal racks 34 are mounted within the housing 14 such that part of the handles 38 are suspended above the fluid volume, while the sheets 50 are submerged in the fluid volume. In the illustrated embodiment, the gripping part 42 is disposed above the fluid volume and the clamping mechanism 46 is submerged in the fluid volume. In other embodiments, other parts of the handle 38 may be disposed above/below the fluid volume. Additionally, depending on the number of sheets 50 and the size of the container 10, the racks 34 are oriented within the housing 14 so the receptacles 36 and handles 38 are sufficiently spaced from each other to prevent any of the sheets 50 from tangling.

Similarly, the size of the racks 34 can be tailored according to the size of the container. For example, FIG. 3 illustrates different sized racks 34 each having a different number of receptacles 36 that can be implemented into different sized containers.

FIG. 6 illustrates another embodiment of a cooling apparatus with like components and features of the cooling apparatus 10 plus “100.” In the illustrated embodiment, the cooling apparatus is an insulative container 110, such as a cooler. The container 110 includes a main body or housing 114 for selectively receiving a plurality of racks 134, and a cover 130 pivotably coupled to the housing 114. In other embodiments, the container 110 can include greater or fewer numbers of racks 134. Each of the racks 134 can be removably coupled to internal sidewalls 126 of the housing 114 or a base 118 of the housing 114 via an interlocking system (not shown). In the illustrated embodiment, the interlocking system includes a plurality of cut-outs on the internal sidewalls 126 of the housing 114 that receive coinciding protrusions extending from the racks 134. In other embodiments, the interlocking system may be other quick-connect systems, such as snap-fit features, hooks, straps, magnets, suction cups, and the like.

Each of the racks 134 includes a plurality of receptacles 136 for retaining a handle 138 in a substantially upright position. Each of the handles 138 includes a gripping part 142 configured to be graspable by a user, and a clamping mechanism 146 used to grip a sheet 50 similar to the cooling apparatus 10. Additionally, like the racks 34 of the cooling apparatus 10, the racks 134 are customizable in size depending on the size of the container to maintain the sheets 50 within the fluid volume.

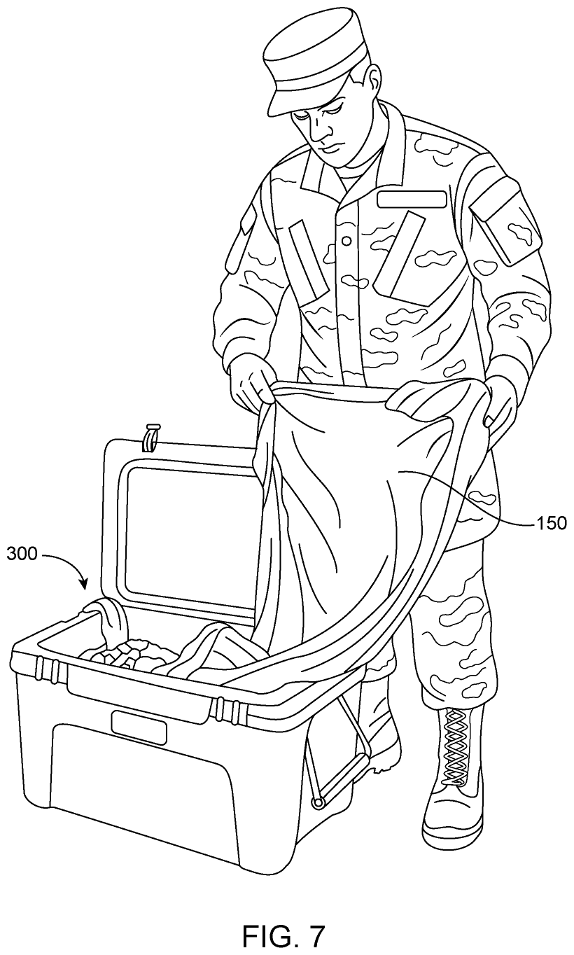

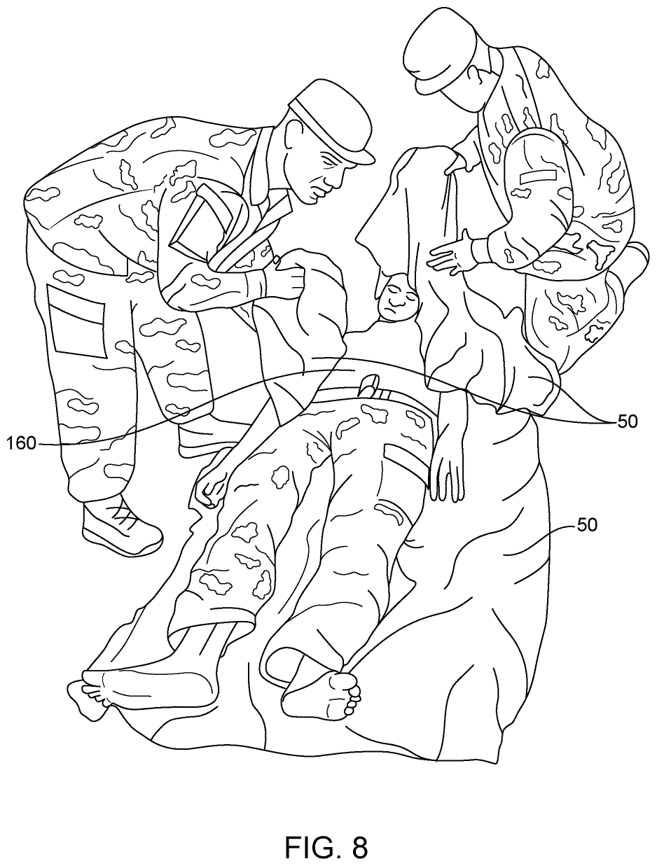

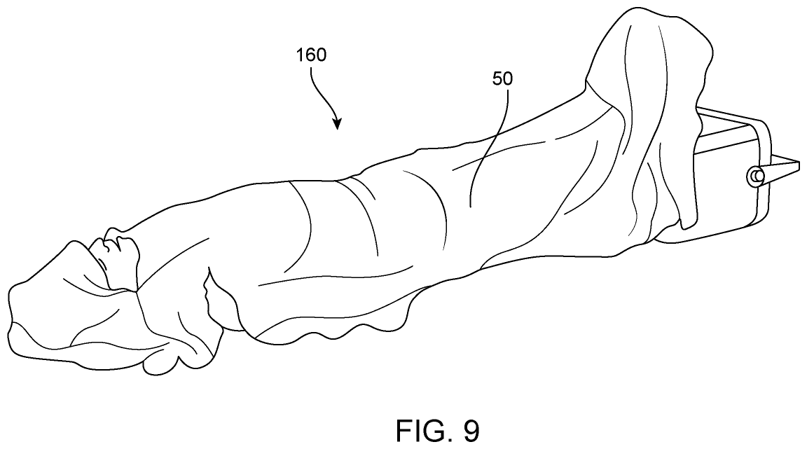

With reference to FIGS. 1, 6, 8 and 9, to use the cooling apparatus 10, 110, a user transports the apparatus 10, 110 to where a patient 160 is overheating. Next, the user grasps one of the handles 38, 138 and removes the handle 38, 138 from the receptacle 36, 136 to pull out the selected sheet 50 secured thereto. Then, the user proceeds to wrap the overheating patient 160 with the cooled sheet 50. In contrast to other methods that rely on the usage of conventional coolers, such as the cooler 300, the cooling apparatus 10, 110 provides a more rapid deployment of sheets 50 to cool the overheating patient 160. Other methods, like those illustrated in FIG. 7, rely on the users to blindly search for sheets 150 submerged in water within the cooler 300. This method causes the sheets 150 to tangle easily resulting in improper deployment. In contrast, the cooling apparatus 10, 110 provides a more uniform, repeatable process for the deployment of cooling sheets 50 to help overheating patients and reduce the number of tangled sheets.

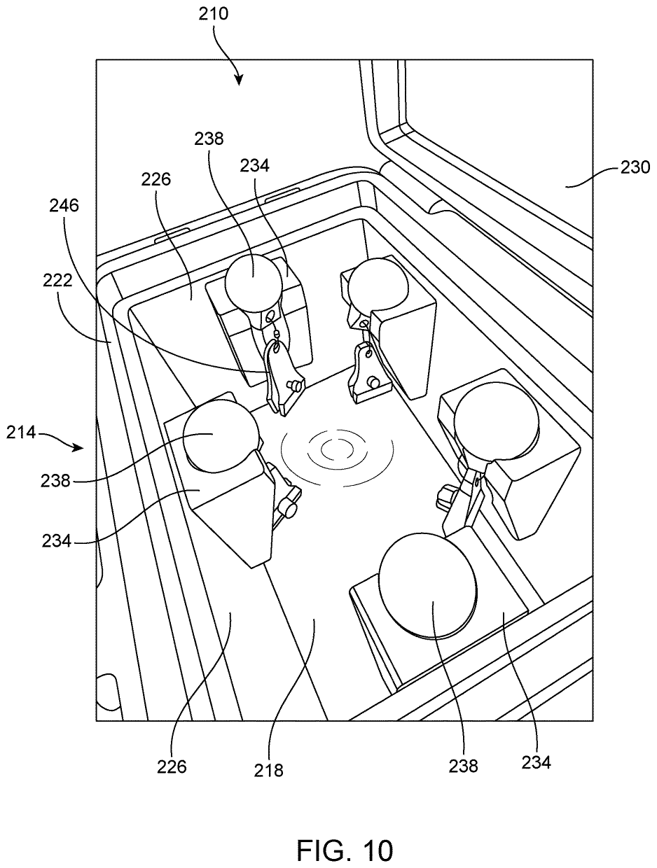

FIGS. 10-13 illustrate another embodiment of a cooling apparatus with like components and features of the cooling apparatus 10 plus “200.” The cooling apparatus 210 functions similarly to the cooling apparatuses 10, 110 described above and will not be re-described here for sake of brevity. In the illustrated embodiment, the cooling apparatus is an insulative container 210, such as a cooler. The container 210 includes a main body or housing 214 having a base 218 for supporting the container 210, an upper side 222 opposite the base 218, and sidewalls 226. The container 210 also includes a cover 230 pivotably coupled to the upper side 222 of the housing 214. In other embodiments, the cover 230 is coupled to the upper side 222 of the housing 214 in other manners.

The housing 214 is configured to support a plurality of racks 234. In the illustrated embodiment, the racks 234 can be removably coupled to the sidewalls 226 of the housing 214 via an interlocking system or an adhesive fastener. The illustrated racks 234 are positioned within the housing 214 such that one or more racks 234 are coupled to and supported by any one of the sidewalls 226 of the housing 214. For example, in the illustrated embodiment, three of the sidewalls 226 of the housing 214 include a single rack 234, while one of the sidewalls 226 includes two racks 234. In other embodiments, the racks 234 may be positioned within the housing 214 in other configurations. In other embodiments, the racks 234 are integrated with the housing 214. In the illustrated embodiment, the interlocking system includes a plurality of cut-outs on the sidewalls 226 of the housing 214 configured to receive corresponding protrusions extending from the racks 234. In other embodiments, the interlocking system may be other quick-connect systems, such as snap-fit features, hooks, straps, magnets, suction cups, and the like. In other embodiments, an adhesive, such as adhesive substrate, can be used to secure the plurality of racks 234 to the sidewalls 226.

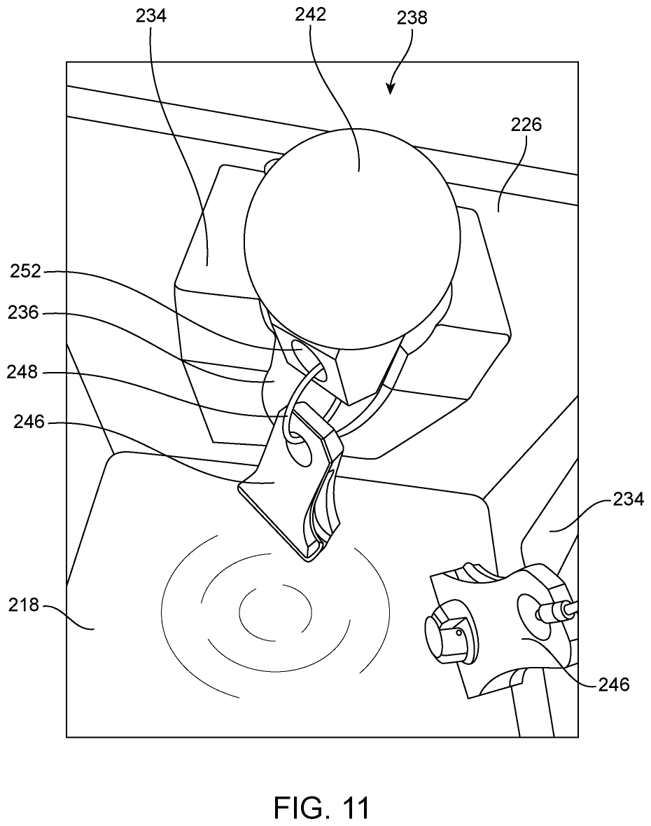

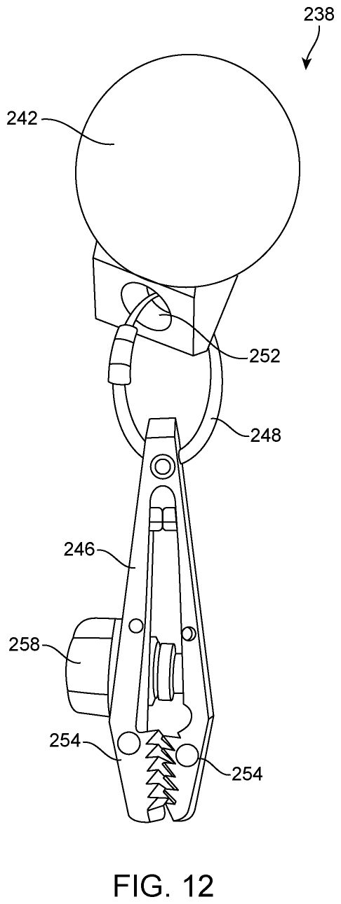

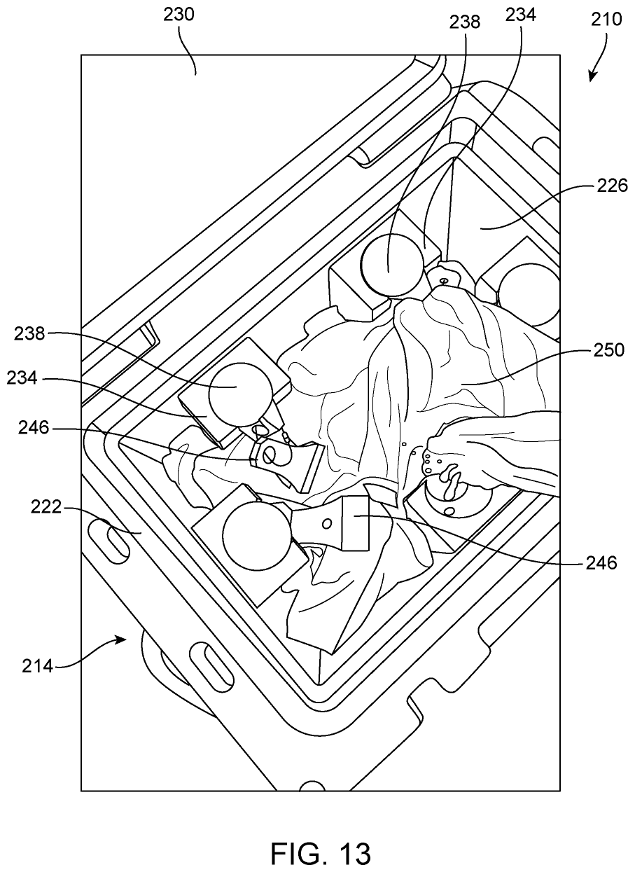

With reference to FIGS. 11-12, each of the illustrated racks 234 includes a receptacle 236 configured to receive and support a handle 238 on the rack 234. The receptacles 236 are shaped to retain the handle 238 in an upright position substantially perpendicular to the base 218 of the housing 214. This orientation ensures the handle 238 remains in a consistent position during transport and use of the apparatus to prevent any sheets 250 (FIG. 13) from getting tangled. In some embodiments, the handles 238 are retained in respective receptacles 236 via a snap-fit connection. In other embodiments, the cover 230 of the container 210 can include cut-outs and/or protrusions shaped to retain the handles 238 within the receptacles 236 during transport of the apparatus.

Each of the handles 238 includes a gripping part 242 configured to be graspable by a user, and a clamping mechanism 246 for selectively gripping a sheet 250 (FIG. 13). In the illustrated embodiment, the gripping part 242 is substantially spherical in shape. In other embodiments, the gripping part 242 may be other shapes. Each of the illustrated handles 238 also includes an attachment member 248 coupling the gripping part 242 to the clamping mechanism 246. The attachment member 248 extends through an aperture 252 formed in the gripping part 242. In the illustrated embodiment, the attachment member 248 is a flexible metal strand with fasteners that can engage each other to form a flexible ring. In other embodiments, the attachment member 248 may be a clip or attachment members, such as carabiners, straps, and the like.

With continued reference to FIGS. 11-12, the illustrated clamping mechanism 246 for each of the handles 238 includes a plurality of jaws 254 and a fastener 258. The fastener 258 is configured to selectively loosen and tighten the jaws 254. For example, in the illustrated embodiment, the fastener 258 is a threaded fastener configured to threadably engage the jaws 254 to selectively open and close the jaws 254. In other embodiments, the fastener 258 can loosen or tighten the jaws 254 in other manners. In yet other embodiments, the clamping mechanism 246 is a clip, such as a tarp clip or a cloth clip.

Returning to FIGS. 11-13, like the cooling apparatus 10, the container 210 is configured to be filled with a volume of fluid (e.g., water) and the racks 234 are mounted within the housing 214 so that part of the handles 238 are above the fluid volume, while the sheets 250 are submerged in the fluid volume. In the illustrated embodiment, the gripping part 242 is disposed above the fluid volume and the clamping mechanism 246 is submerged in the fluid volume. In other embodiments, other parts of the handle 238 may be disposed above/below the fluid volume. Additionally, depending on the number of sheets 250 and the size of the container 210, the racks 234 are oriented within the housing 214 so the receptacles 236 and handles 238 are sufficiently spaced from each other to prevent any of the sheets 250 from tangling.

In some embodiments, each of the clamping mechanisms 246 of respective handles 238 can be oriented to sit on top of the sheets 250 to facilitate easy removal from the container 210 and simultaneously prevent the sheet 250 from pushing the handles 238 off of the receptacle 236 during transport of the apparatus.

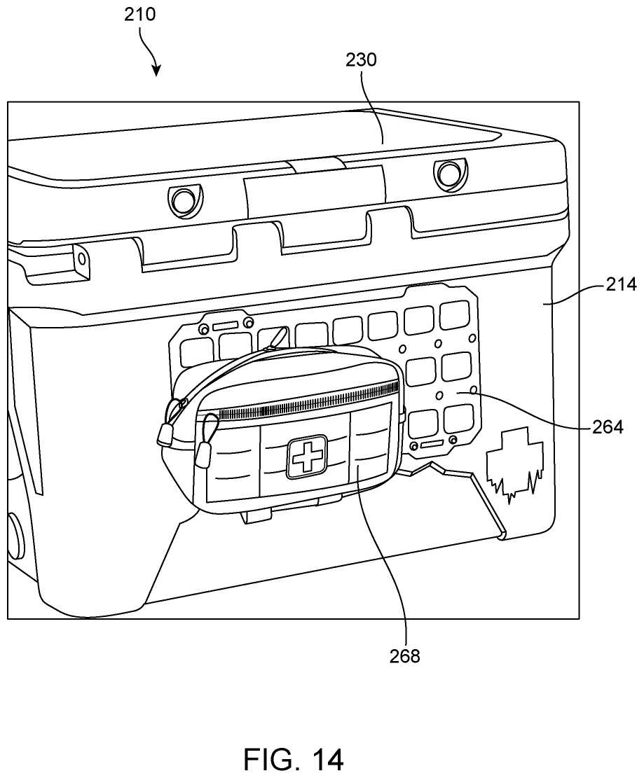

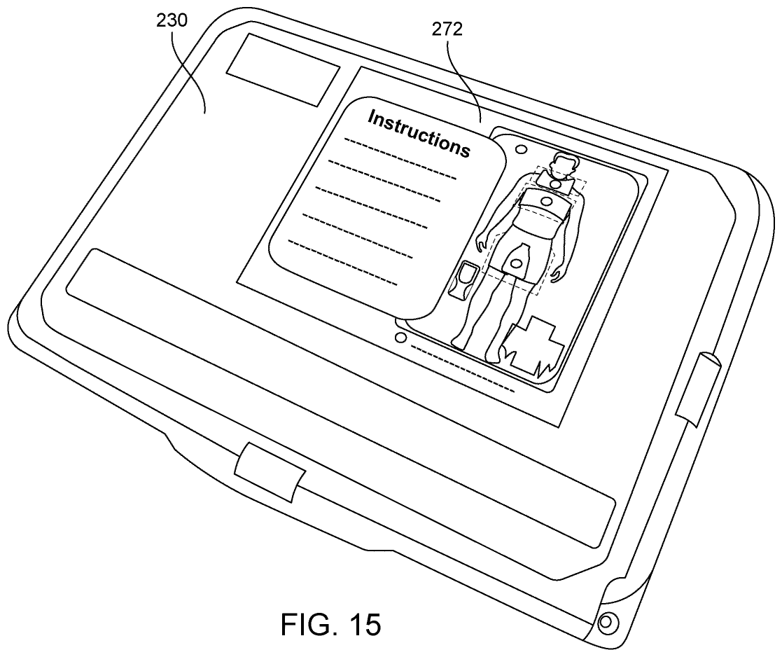

With reference to FIGS. 14 and 15, in other embodiments, the cooling apparatus 210 can include a mount 264 coupled to the housing 214. The illustrated mount 264 is an attachment panel for coupling medical accessories 268 (e.g., medical pouches) or other field accessories (e.g., thermometers, etc.). In some embodiments, the attachment panel is compatible with modular lightweight load-carry equipment (MOLLE)/pouch attachment ladder systems (PALS). The cooling apparatus 210 can also include instructional graphics 272 printed anywhere on the apparatus 210 that outline a step-by-step method of using the apparatus 210 and deploying the sheets 250 to treat a patient. In the illustrated embodiment, the graphics 272 are printed on the cover 230. In some embodiments, the graphics 272 can be affixed to the apparatus 210 by an adhesive. In other embodiments, the graphics 272 may be printed in other locations inside or outside of the apparatus 210, or affixed to the apparatus 210 with other known methods.

Although the disclosure has described in detail certain preferred embodiments, variations and modifications exist within the scope and spirit of one or more independent aspects of the invention as described.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

Various features of the invention are set forth in the following claims. These and other modifications and variations to the present subject matter may be practiced by those of ordinary skill in the art, without departing from the spirit and scope of the present subject matter, which is more particularly set forth herein above and any appending claims. In addition, it should be understood the aspects of the various embodiments may be interchanged either in whole or in part. Furthermore, those of ordinary skill in the art will appreciate that the foregoing description is by way of example only, and is not intended to limit the present subject matter.

What is claimed is:

1. A cooling apparatus comprising:
a housing;
a rack supported within the housing, the rack including a receptacle; and
a handle removably coupled to the receptacle, the handle comprising a clamping mechanism for selectively clamping a fabric.

2. The cooling apparatus of claim 1, wherein the housing is an insulative housing, and wherein the insulative housing is configured to house a volume of fluid.

3. The cooling apparatus of claim 2, wherein the fabric is a fabric sheet, and wherein the fabric sheet is submerged in the volume of fluid.

4. The cooling apparatus of claim 2, wherein part of the handle is located above the volume of fluid.

5. The cooling apparatus of claim 1, wherein the rack is removably coupled to the housing by an interlocking system.

6. The cooling apparatus of claim 5, wherein the housing further includes a base configured to support the cooling apparatus, and a plurality of sidewalls, and wherein the interlocking system includes a cut-out formed on one of the sidewalls and a corresponding protrusion formed on the rack.

7. The cooling apparatus of claim 6, wherein the receptacle retains the handle in an upright position substantially perpendicular to the base of the housing.

8. The cooling apparatus of claim 1, wherein the clamping mechanism includes a plurality of jaws and a fastener configured to selectively loosen and tighten the jaws.

9. The cooling apparatus of claim 1, wherein the handle includes a gripping portion configured to be graspable by a user, the gripping portion being coupled to the clamping mechanism and being receivable in the receptacle.

10. The cooling apparatus of claim 9, wherein the handle further comprises an attachment member coupling the clamping mechanism to the gripping portion.

11. The cooling apparatus of claim 1, wherein the housing includes a mount configured to receive an accessory.

12. A rack system for use with an insulative container, the rack system comprising:
one or more racks selectively mountable within a housing of the insulative container, the one or more racks each including one or more receptacles; and
one or more handles each selectively mountable to the one or more receptacles, each of the one or more handles comprising a clamping mechanism for selectively clamping a fabric.

13. The rack system of claim 12, wherein each of the one or more racks is selectively mountable to a sidewall of the housing.

14. The rack system of claim 12, wherein the clamping mechanism includes a plurality of jaws and a fastener configured to selectively loosen and tighten the jaws.

15. The rack system of claim 12, wherein the one or more handles each includes a gripping portion configured to be graspable by a user and receivable in one of the receptacles, the gripping portion being coupled to the clamping mechanism.

16. The rack system of claim 15, wherein the gripping portion is substantially spherical in shape.

17. A cooling apparatus comprising:
a housing comprising a base configured to support the cooling apparatus, and a plurality of sidewalls;
a plurality of racks, each of the racks being supported by the sidewalls and including a receptacle; and
a plurality of handles removably coupled to each of one the receptacles, each one of the handles comprising a clamping mechanism for selectively clamping a fabric.

18. The cooling apparatus of claim 17, wherein each one of the sidewalls supports one of the racks.

19. The cooling apparatus of claim 17, wherein the clamping mechanism includes a plurality of jaws and a fastener configured to selectively loosen and tighten the jaws.

20. The cooling apparatus of claim 17, wherein the housing is an insulative housing configured to house a volume of fluid, and wherein the fabric is a fabric sheet submerged in the volume of fluid.
